(19) United States
(12) Patent Application Publication
(10) Pub. No.: US 2024/0066224 A1
(43) Pub. Date: Feb. 29, 2024

(54) USE OF LOGARITHMIC TRANSFORM/FILTER TO IMPROVE OPERATION OF MEDICAMENT DELIVERY DEVICE

(71) Applicant: INSULET CORPORATION, Acton, MA (US)

(72) Inventors: Yibin ZHENG, Hartland, WI (US); Joon Bok LEE, Acton, MA (US); Jason O’CONNOR, Acton, MA (US)

(21) Appl. No.: 18/451,530

(22) Filed: Aug. 17, 2023

Related U.S. Application Data
(60) Provisional application No. 63/374,042, filed on Aug. 31, 2022.

(51) Int. Cl. A61M 5/172

(52) U.S. Cl. CPC A61M 5/1723; A61M 2202/0486; A61M 2205/50

(57) ABSTRACT

Exemplary embodiments may apply a transform or filter to analyte level values of the users to make the analyte level values conform with a normal distribution that is symmetric relative to the mean. The transformed or filtered analyte level values may be used by the control system of a medicament delivery device in determining medicament delivery doses. In some embodiments, the medicament is insulin, and the analyte level is a glucose level of a user. In such instances, a logarithmic filter or transform may be applied to the glucose level readings of the user.

### RELATED APPLICATIONS

This application claims the benefit of U.S. Provisional Patent Application No. 63/374,042, filed Aug. 31, 2022, the entire contents of which are incorporated herein by reference in their entirety.

### BACKGROUND

Many conventional medicament delivery devices employ control systems that attempt to keep an analyte level of a user at a target analyte level. For example, many conventional insulin delivery devices seek to keep a user's glucose level at a target glucose level. These conventional control systems gather glucose level readings of the user on an ongoing basis and compare the glucose level readings with the target glucose level. Based at least in part on the difference between the glucose level readings and the target glucose level, the conventional insulin delivery device makes decisions as to how much insulin to deliver to the user. Other factors also may play a role.

The conventional insulin delivery devices may employ a glucose cost function that assigns a cost to glucose excursions that will exist in the future if a given dose of insulin is delivered to the user at the present time. A glucose excursion is an instance where the user's glucose level deviates from the target glucose level for a non-negligible period of time. The aim of the control system is to reduce such glucose excursions. The control systems of such conventional insulin delivery devices may apply optimization algorithms to choose an insulin dose that has the lowest glucose cost and then deliver the chosen lowest glucose cost insulin dose to the user.

One challenge with such conventional analyte cost functions (e.g., glucose cost functions) is that the conventional analyte cost functions may presuppose that the analyte levels of users that are used by the control systems to determine medicament doses for the users follow a Gaussian or normal distribution and are symmetric around a mean value. Unfortunately, the actual distribution of analyte levels for the users may be non-Gaussian and asymmetric. As a result, the control systems of such conventional medicament delivery devices may not perform as well as desired.

### SUMMARY

In accordance with an inventive facet, an insulin delivery device for delivering insulin to a user includes a non-transitory computer-readable storage medium storing computer programming instructions and a processor for executing the computer programming instructions. Executing the computer programming instructions causes the processor to determine an insulin dose to be delivered to the user with the insulin delivery device that has a lowest glucose cost. The glucose cost is determined based on effective glucose level values for the user, and the effective glucose level values are determined by applying a logarithmic transform to predicted glucose level values for a time horizon. The execution of the computer programming instructions also causes the insulin dose to be delivered by the insulin delivery device to the user.

The predicted glucose level values may be predicted from previous glucose level values of the user of earlier cycles or times and/or from predicted glucose level values for the user for earlier cycles or times when previous glucose level values are not yet available. The non-transitory computer-readable storage medium may store a glucose level history of the user, and the glucose level values of the user for points in time before when the predicting occurs may be part of the glucose level history. The insulin delivery device may include an insulin reservoir for holding insulin for delivery to the user. The computer programming instructions, when executed by the processor, may cause the processor to initiate delivery of the insulin dose from the reservoir to the user. The processor in applying a logarithmic transform to predicted glucose level values for the time horizon may apply a logarithmic function to the predicted glucose level values. The applying of the logarithmic transform may entail, for each of the predicted glucose level values, applying a logarithmic function to a product of a scaling factor and a ratio of the predicted glucose level value to the set point glucose level value to yield a logarithmic value. The applying of the logarithmic transform may include multiplying the logarithmic value by the set point glucose level value to produce one of the predicted logarithmic glucose level values. The glucose cost may be a product of a coefficient and a sum of squared differences between each of the projected glucose level values and the set point glucose level value over the time horizon.

In accordance with another inventive facet, an insulin delivery system for delivering insulin to a user includes a non-transitory computer-readable storage medium storing computer programming instructions and a processor for executing the computer programming instructions. The computer programming instructions for causing the processor to choose a selected one of candidate insulin dosages that has a best cost value when a cost function is applied to the candidate insulin dosages for delivery by the insulin delivery device to the user. The cost function includes an insulin cost component and a glucose cost component. The glucose cost component is based on cumulative differences between predicted effective glucose level values and set point glucose level values over a time horizon. The predicted effective glucose level values are determined by applying a logarithmic function to predicted glucose level values of the user over the time horizon. The computer programming instructions include computer programming instructions to cause delivery of the selected one of the candidate insulin dosages to the user.

At least one of the predicted glucose level values may be determined by the processor from glucose level values of the user for points in time before the time horizon. The insulin delivery system may include an insulin reservoir for holding insulin. The computer programming instructions, when executed by the processor, may cause the processor to initiate delivery of the selected one of the candidate insulin dosages from the reservoir to the user. The predicted glucose level values may be determined by the processor from glucose level values before the time horizon and/or predicted glucose level values when previous glucose level values are not yet available. Each of the predicted effective glucose level values may be determined by applying a logarithmic function to a product of a scaling factor and a ratio of a corresponding predicted glucose level value to one of the set point glucose level values to yield a logarithmic value. The computer programming instructions, when executed by the processor, may further cause the processor to multiply the logarithmic value by the set point glucose level value to produce one of the predicted logarithmic projected glucose level values. The glucose cost value may be a product of a coefficient and a sum of squared differences between each of the projected glucose level values and the set point glucose level values over the time horizon. The insulin delivery system may include other components, such as a smartphone or one or more sensors, in some exemplary embodiments.

In accordance with a further inventive facet, an insulin delivery device includes a non-transitory computer-readable storage medium storing computer programming instructions and a processor for executing the computer programming instructions. The computer programming instructions cause the processor to control automated insulin delivery (AID) to a user by the insulin delivery device. The control includes transforming a glucose level value of the user into a logarithmic glucose level value; using the logarithmic glucose level value in determining what insulin dosage is to be delivered to the user as part of the AID by the insulin delivery device; and causing the determined insulin dosage to be delivered to the user from the insulin delivery device.

The determined insulin dosage may be for basal delivery to the user. The transforming may include applying a logarithmic function to the glucose level value.

### BRIEF DESCRIPTION OF THE DRAWINGS

FIG. 1 depicts an illustrative medicament delivery system of an exemplary embodiment for delivering medicament to a user.

FIG. 2 depicts an illustrative control loop for the medicament delivery system of the exemplary embodiments.

FIG. 3 depicts a flowchart of illustrative steps that may be performed by the control loop in exemplary embodiments.

FIG. 4A depicts a histogram of densities for estimated glucose values (EGV's) for a population with a normal fit overlay.

FIG. 4B depicts a curve of cumulative probability of EGV's for the population with an overlay of a corresponding curve of cumulative probability for a Gaussian distribution.

FIG. 5A depicts inputs and an output for a conventional control system of an insulin delivery device.

FIG. 5B depicts inputs and an output for control system of an insulin delivery device in accordance with exemplary embodiments.

FIG. 6A depicts a histogram of densities for EGV's for a population and a log normal fit overlay.

FIG. 6B depicts a curve of cumulative probability of EGV's for the population with an overlay of a corresponding curve of cumulative probability of EGV's for a log normal distribution.

FIG. 7A depicts a flowchart of illustrative steps that may be performed in exemplary embodiments to determine an insulin dose for a cycle.

FIG. 7B depicts a histogram of densities for effective glucose values G_{eff}(i) for a population with a normal fit overlay.

FIG. 7C depicts a curve of cumulative probability for G_{eff}(i) values for the population with an overlay of a corresponding curve of cumulative probability for G_{eff}(i) values for a normal distribution.

FIG. 8 depicts a flowchart of illustrative steps that may be performed in exemplary embodiments to calculate G_{eff}(i).

FIG. 9 depicts a flowchart of illustrative steps that may be performed in exemplary embodiments to determine total cost of a candidate insulin dose using G_{eff}(i).

FIG. 10 depicts a flowchart of illustrative steps that may be performed in exemplary embodiments to determine glucose cost.

FIG. 11 depicts a flowchart of illustrative steps that may be performed in exemplary embodiments to determine G_{eff}(i) for a future cycle.

### DETAILED DESCRIPTION

Exemplary embodiments recognize that analyte levels of users of medicament delivery devices that are used by the control systems of the medicament delivery devices to influence medicament deliveries may not conform to a Gaussian distribution and may not be symmetric around a mean. For example, the exemplary embodiments described herein recognize that the distribution of glucose level readings across a population conforms with a log normal distribution and not a Gaussian (“normal”) distribution. Exemplary embodiments may apply a transform (such as logarithmic function) or filter to analyte level values of the users to make the analyte level values conform with a normal distribution that is symmetric relative to the mean. The transformed or filtered analyte level values may be used by the control system of a medicament delivery device in determining medicament delivery doses. In some embodiments, the medicament is insulin, and the analyte level is a glucose level of a user. In such instances, a logarithmic filter or transform may be applied to the glucose level readings of the user.

In some exemplary embodiments, the medicament delivery device may be an insulin delivery device, such as a patch insulin pump that is worn on the user. In such embodiments, the control system of the insulin delivery device may use a glucose cost function to determine the cost of glucose excursions of candidate insulin doses. In the exemplary embodiments, the glucose cost function may calculate cost based on a logarithm of a current glucose level of the user. The logarithm of the current glucose level exhibits a Gaussian distribution of values across a population of users. Use of the logarithmic values rather than the raw glucose level readings may improve the performance of the insulin delivery device. Simulation results indicate a 1% to 2% improvement of time in a desired range for users when the described approach is used.

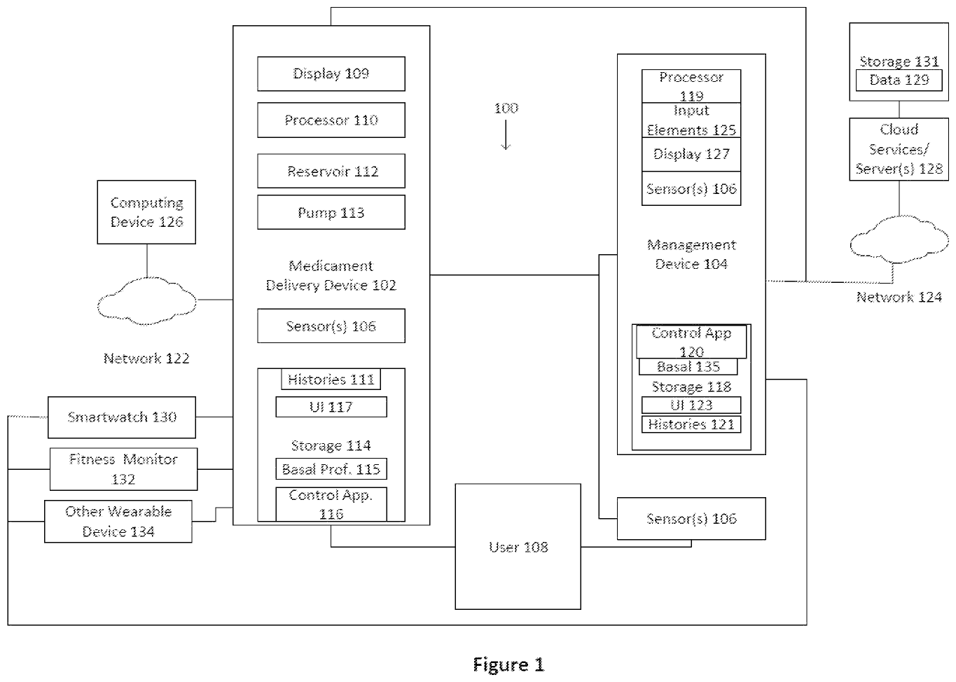

FIG. 1 depicts an illustrative medicament delivery system 100 that is suitable for delivering a medicament to a user 108 in accordance with the exemplary embodiments. The medicament delivery system 100 includes a medicament delivery device 102. The medicament delivery device 102 may be a wearable device that is worn on the body of the user 108 or carried by the user. The medicament delivery device 102 may be directly coupled to a user (e.g., directly attached to a body part and/or skin of the user 108 via an adhesive or the like) or carried by the user (e.g., on a belt or in a pocket) with the medicament delivery device 102 being connected to an infusion site where the medicament is injected using a needle and/or cannula. In a preferred embodiment, a surface of the medicament delivery device 102 may include an adhesive to facilitate attachment to the user 108.

The medicament delivery device 102 may include a processor 110. The processor 110 may be, for example, a microprocessor, a logic circuit, a field programmable gate array (FPGA), an application specific integrated circuit (ASIC) or a microcontroller. The processor 110 may maintain a date and time as well as other functions (e.g., calculations or the like). The processor 110 may be operable to execute a control application 116 encoded in computer programming instructions stored in the storage 114 that enables the processor 110 to direct operation of the medicament delivery device 102. The control application 116 may be a single program, multiple programs, modules, libraries, or the like. The control application may be responsible for implementing the control loop that provides feedback and adjustments to medicament dosages (i.e., when deliveries are made and what doses of medicament are delivered). The processor 110 also may execute computer programming instructions stored in the storage 114 for a user interface 117 that may include one or more display screens shown on display 109. The display 109 may display information to the user 108 and, in some instances, may receive input from the user 108, such as when the display 109 is a touchscreen.

The control application 116 may control delivery of a medicament to the user 108 per a control approach like that described herein. The storage 114 may hold histories 111 for a user, such as a history of basal deliveries, a history of bolus deliveries, and/or other histories, such as a meal event history, exercise event history, analyte level history, such as glucose level history, and/or the like. The storage 114 also may include one or more basal profiles 115 that are used when the medicament delivery device is operating in open loop mode. In addition, the processor 110 may be operable to receive data or information. The storage 114 may include both primary memory and secondary memory. The storage 114 may include random access memory (RAM), read only memory (ROM), optical storage, magnetic storage, removable storage media, solid state storage or the like.

The medicament delivery device 102 may include a tray or cradle and/or one or more housings for housing its various components including a pump 113, a power source (not shown), and a reservoir 112 for storing a medicament for delivery to the user 108. A fluid path to the user 108 may be provided, and the medicament delivery device 102 may expel the medicament from the reservoir 112 to deliver the medicament to the user 108 using the pump 113 via the fluid path. The fluid path may, for example, include tubing coupling the medicament delivery device 102 to the user 108 (e.g., tubing coupling a cannula to the reservoir 112) and may include a conduit to a separate infusion site.

There may be one or more communications links with one or more devices physically separated from the medicament delivery device 102 including, for example, a management device 104 of the user and/or a caregiver of the user, a sensor 106, a smartwatch 130, a fitness monitor 132 and/or another variety of wearable device 134. The communication links may include any wired or wireless communication links operating according to any known communications protocol or standard, such as Bluetooth®, Wi-Fi, a near-field communication standard protocol, a cellular standard protocol, or any other wireless protocol.

The medicament delivery device 102 may interface with a network 122 via a wired or wireless communications link. The network 122 may include a local area network (LAN), a wide area network (WAN) or a combination therein. A computing device 126 may be interfaced with the network, and the computing device may communicate with the medicament delivery device 102.

The medicament delivery system 100 may include one or more sensor(s) 106 for sensing the levels of one or more analytes. The sensor(s) 106 may be coupled to the user 108 by, for example, adhesive or the like and may provide information or data on one or more medical conditions and/or physical attributes of the user 108. The sensor(s) 106 may be physically separate from the medicament delivery device 102 or may be an integrated component thereof. In some embodiments, the sensor(s) 106 may include a glucose sensor, such as a continuous glucose monitor (CGM). The sensor(s) 106 may sense other analyte levels, such as, for example, heart rate, breathing rate, temperature, elevation, movement, perspiration, ketone levels, blood oxygen, alcohol level, level of drugs in blood, etc.

The medicament delivery system 100 may include a management device 104. In some embodiments, a management device is not needed as the medicament delivery device 102 may manage itself. The management device 104 may be a special purpose device, such as a dedicated personal diabetes manager (PDM) device. The management device 104 may be a programmed general-purpose device, such as any portable electronic device including, for example, a dedicated controller, such as a processor, a microcontroller, or the like. The management device 104 may be used to program or adjust operation of the medicament delivery device 102 and/or the sensor(s) 106. The management device 104 may be any portable electronic device including, for example, a dedicated device, a smartphone, a smartwatch or a tablet. In the depicted example, the management device 104 may include a processor 119 and a storage 118. The processor 119 may execute processes to manage a user's analyte levels and to control the delivery of the medicament to the user 108. The medicament delivery device 102 may provide data from the sensor(s) 106 and other data to the management device 104. The data may be stored in the storage 118. The processor 119 may also be operable to execute programming code stored in the storage 118. For example, the storage 118 may be operable to store a control application 120 for execution by the processor 119. The control application 120 may be responsible for controlling the medicament delivery device 102, such as by controlling the AID delivery of insulin to the user 108. The storage 118 may store the control application 120, histories 121 like those described above for the medicament delivery device 102, one or more basal profiles 135 and other data and/or programs.

A display 127, such as a touchscreen, may be provided for displaying information. The display 127 may display user interface (UI) 123 for interacting with the user. The display 127 also may be used to receive input, such as when the display is a touchscreen. The management device 104 may further include input elements 125, such as a keyboard, button, knobs, or the like, for receiving input form the user 108.

The management device 104 may interface with a network 124, such as a LAN or WAN or combination of such networks via wired or wireless communication links. The management device 104 may communicate over network 124 with one or more servers or cloud services 128. Data, such as sensor values, may be sent, in some embodiments, for storage and processing from the medicament delivery device 102 directly to the cloud services/server(s) 128 or instead from the management device 104 to the cloud services/server(s) 128. The cloud services/server(s) 128 may provide output from the model 115 as needed to the management device 104 and/or medicament delivery device 102 during operation.

Other devices, like smartwatch 130, fitness monitor 132 and wearable device 134 may be part of the medicament delivery system 100. These devices 130, 132 and 134 may communicate with the medicament delivery device 102 and/or management device 104 to receive information and/or issue commands to the medicament delivery device 102. These devices 130, 132 and 134 may execute computer programming instructions to perform some of the control functions otherwise performed by processor 110 or processor 119, such as via control applications 116 and 120. These devices 130, 132 and 134 may include displays for displaying information. The displays may show a user interface for receiving input by the user, such as to request a change or pause in dosage or to request, initiate, or confirm delivery of a bolus of a medicament, or for displaying output, such as a change in dosage (e.g., of a basal delivery amount) as determined by processor 110 or management device 104. These devices 130, 132 and 134 may also have wireless communication connections with the sensor 106 to directly receive analyte measurement data.

A wide variety of medicaments may be delivered by the medicament delivery device 102. The medicament may be insulin for treating diabetes. The medicament may be glucagon for raising a user's glucose level. The medicament may also be a glucagon-like peptide (GLP)-1 receptor agonists for lowering glucose or slowing gastric emptying, thereby delaying spikes in glucose after a meal. Alternatively, the medicament delivered by the medicament delivery device 102 may be one of a pain relief agent, a chemotherapy agent, an antibiotic, a blood thinning agent, a hormone, a blood pressure lowering agent, an antidepressant, an antipsychotic, a statin, an anticoagulant, an anticonvulsant, an antihistamine, an anti-inflammatory, a steroid, an immunosuppressive agent, an antianxiety agent, an antiviral agents, a nutritional supplement or a vitamin.

The functionality described below for the exemplary embodiments may be under the control of or performed by the control application 116 of the medicament delivery device 102 or the control application 120 of the management device 104. In some embodiments, the functionality may be under the control of or performed by the cloud services or servers 128, the computing device 126 or by the other enumerated devices, including smartwatch 130, fitness monitor 132 or another wearable device 134.

The medicament delivery device 102 may operate in an open loop mode or in a closed loop mode. In the open loop mode, the user 108 manually inputs the amount of medicament to be delivered (such as per hour) for segments of the day. The inputs may be stored in a basal profile 115, 135 for the user 108. In other embodiments, a basal profile may not be used. The control application 116, 120 uses the input information from the basal profile 115, 135 to control basal medicament deliveries in open loop mode. In contrast, in the closed loop mode, the control application 116, 120 determines the medicant delivery amount for the user 108 on an ongoing basis based on a feedback loop. For an insulin delivery device, the aim of the closed loop mode is to have the user's glucose level at a target glucose level. The basal dosages may be delivered at fixed regular intervals, designated as cycles, such as every five minutes. In some embodiments, the cycle may represent a period of time between about 1 min to about 30 min, more specifically between about 2 min to about 15 min, and in particular between about 3 min to 10 min.

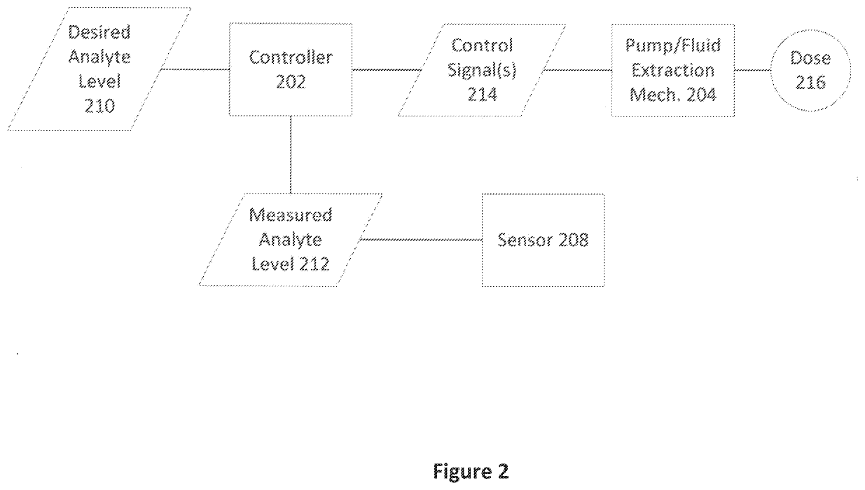

As was mentioned above, a control loop may be provided to adjust a basal delivery dose of medicament based on current analyte level readings, such as glucose level readings, for example. FIG. 2 illustrates a simplified block diagram of an example of such a control loop 200 suitable for practicing an exemplary embodiment. The example control loop 200 may include a controller 202, a pump mechanism or other fluid extraction mechanism 204 (hereinafter “pump 204”), and a sensor 208. The controller 202 may be part of control application 116 or 120. The controller 202, pump 204, and sensor 208 may be communicatively coupled to one another via a wired or wireless communication paths. The sensor 208 may be a glucose monitor in some exemplary embodiments, such as, for example, a CGM. The sensor 208, for example, may be operable to measure glucose level values of a user to generate the measured analyte level 212. In some embodiments, the sensor 208 may apply a logarithmic filter or transform to the analyte level reading and forward the resulting value to the control application 116 or 120, such as via a wireless connection.

As shown in the example, the controller 202 may receive a desired analyte level 210, indicating a desired analyte level or range for a user. The desired analyte level 210 may be received from a user interface to the controller 202 or other device or by an algorithm that automatically determines a desired analyte level 210 for a user. The sensor 208 may be coupled to the user and be operable to measure an approximate value of an actual analyte level of the user. For cases where the analyte level is a glucose level, it is worth noting that the measured glucose level is only an approximate value of a user's glucose level. There may be errors in the measured glucose levels. The errors may, for example, be attributable to factors, such as age of the sensor 208, location of the sensor 208 on a body of a user, environmental factors (e.g., altitude, humidity, barometric pressure), or the like. These measured glucose levels may be referred to as “estimated glucose values” (EGV's). In response to the measured analyte level or value, the sensor 208 may generate a signal indicating the measured analyte level 212. The controller 202 may receive from the sensor 208 via a communication path the measured analyte level signal 212.

Based on the desired analyte level signal 210 and the measured analyte level signal 212, the controller 202, as will be described below, may calculate a glucose cost using a glucose cost function. In exemplary embodiments, a logarithmic transform may be applied to analyte level values, such as glucose level values, and the transformed values may be used in determining an analyte cost. The medicament dose with the lowest cost may be chosen for delivery by the controller 202. The controller 202 then may generate one or more control signals 214 for directing operation of the pump 204. For example, one of the control signals 214 may cause the pump 204 to deliver a dose of medicament 216 to a user via output 206. The dose of medicament 216 may be determined as an appropriate amount of medicament to drive the actual analyte level of the user toward the desired analyte level. Based on operation of the pump 204 as determined by the control signals 214, the user may receive the dose of medicament 216 from the pump 204.

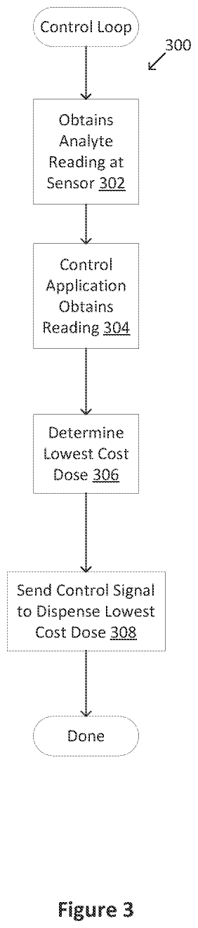

FIG. 3 depicts a flowchart 300 of steps that may be performed by exemplary embodiments in determining what dose of medicament to deliver to the user as part of the closed loop control system. These steps may be performed by processor 110, processor 119 or other components (at least in part), like smartwatch 130, fitness monitor or wearable device 134. That said, for purposes of simplicity below, the discussion simply refers to processor 110. Initially, as was described above relative to FIG. 2, at 302, an analyte level reading is obtained by the sensor 208. At 304, the analyte level reading is sent via a signal 212 to the controller 202.

The control system attempts to minimize the aggregate penalty of a cost function over a range of possible doses as constrained by the control system. At 306, the dose with the best cost function value (e.g., lowest cost) is selected. Depending on how the cost function is configured, the best value may be the lowest value or the highest value. The cost function used in exemplary embodiments will be described in more detail below. The candidate doses are those within the search space of all available doses that conform with the constraints imposed by the control system. For instance, the minimum dose size may be zero or a positive minimum amount. Other constraints may be, for example, the maximum dose size. The control system may apply an optimization strategy to locate the lowest cost candidate dose within the space. Regression strategies may be used in some instances. The term “regression strategies” as used herein may relate to different statistical processes for estimating the relationships between a dependent variables and one or more independent variables, in particular to identify minima or maxima of dependent variables, e.g. glucose cost or insulin cost.

At 308, control signal 214 may be generated by the controller 202 and sent to the pump 204 to cause the pump to deliver the desired medicament dose 216 to the user.

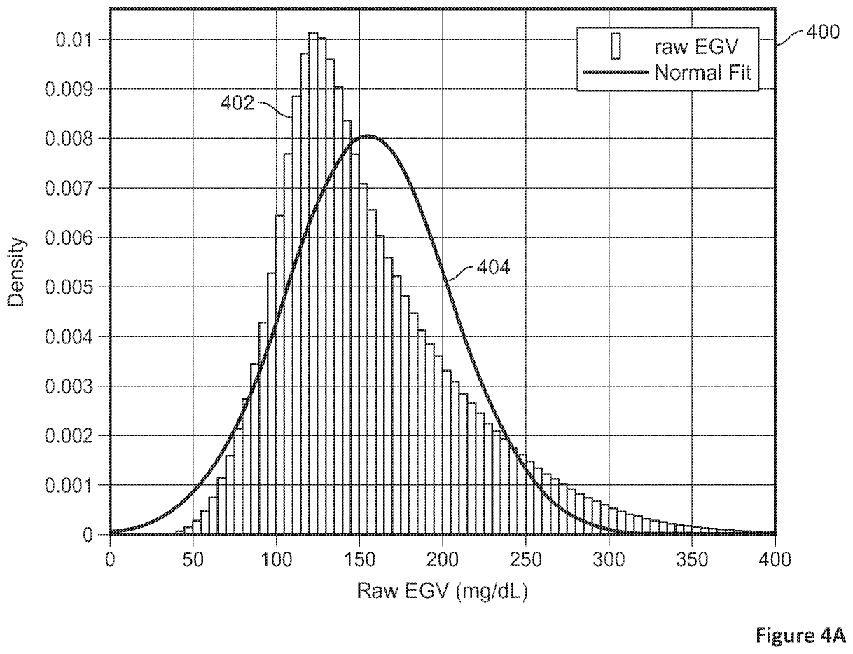

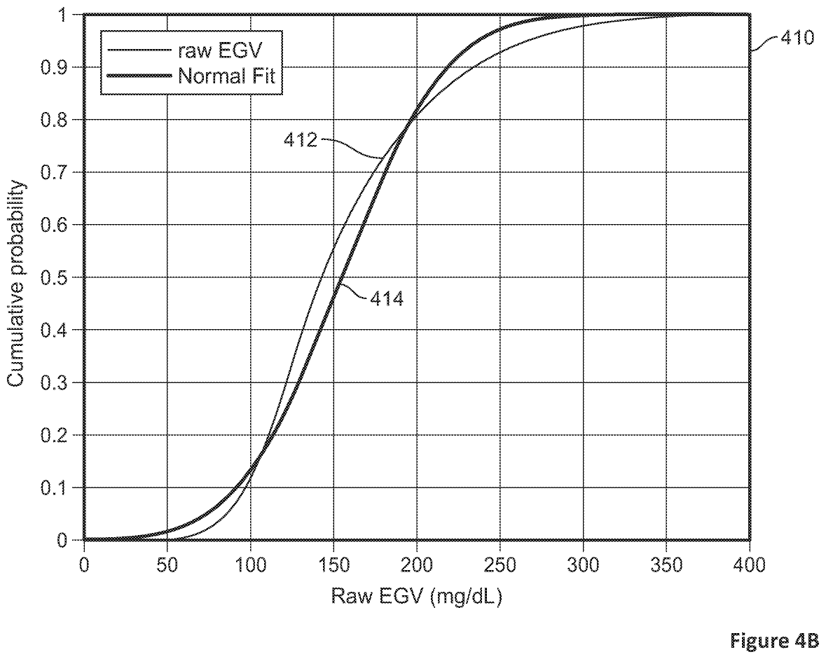

Before delving into the details of the logarithm transform or filter and the cost function, it is helpful to revisit the problem encountered with some conventional medicament delivery devices. FIG. 4A depicts a plot 400 that includes a histogram 402 of the density of various EGV's for a population of users of a particular type of insulin delivery device. Also depicted on the plot 400 is an overlay of an ideal normal distribution curve 404. As can be seen, there are meaningful differences between the histogram 402 and the ideal normal distribution curve 404. The histogram 402 indicates that the actual EGV's for users as reflected by the histogram 402 do not conform with a symmetric distribution. There are more EGV's above the peak value in the histogram than below the peak value. In other words, users typically experience more hyperglycemia than hypoglycemia (e.g., have more blood glucose values above 150 mg/dL than below 70 mg/dL). Moreover, the peak of the histogram 402 does not align with the peak of the ideal normal distribution curve 404.

The difference between the raw EGV's and a Gaussian distribution also can be seen in FIG. 4B, which depicts a plot of the cumulative probability across all raw EGV's in a population. Curve 412 is the curve of the cumulative probabilities to raw EGV's for the population. Curve 414 represents the cumulative probabilities to raw EGV's for a Gaussian distribution (“normal fit”). As can be seen, there, is a disparity between the two curves 412 and 414, indicative of the actual EGV's not conforming to a normal Gaussian distribution.

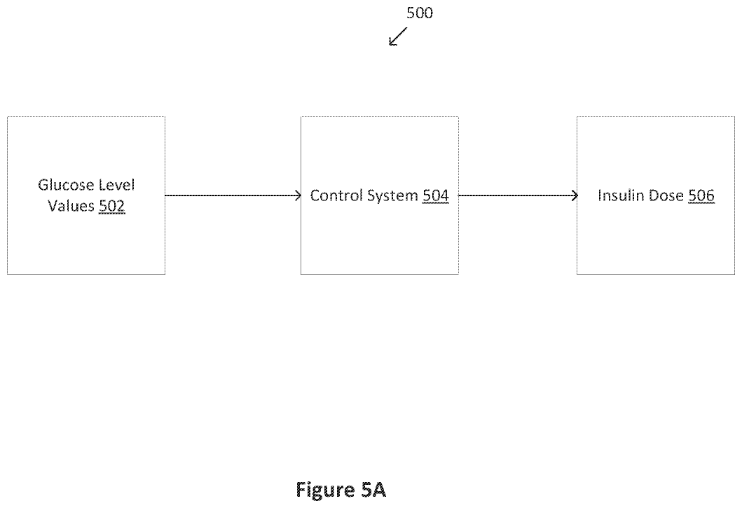

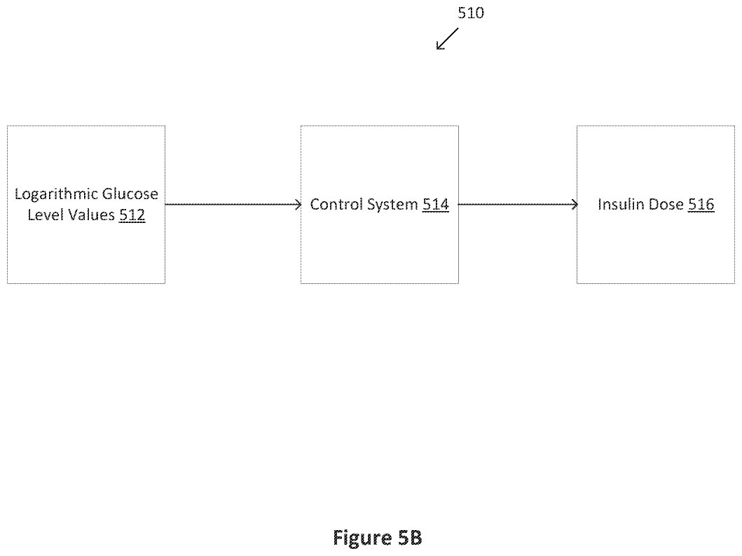

FIG. 5A depicts an illustrative block diagram 500 for a conventional control system 504 of an insulin delivery device. As mentioned above, glucose level values for a user from sensor(s) 106, such as a CGM, are input to a control system 504. The control system 504 uses the glucose level values 504 to determine and output an insulin dose 506. Often, this process may be repeated for each operational cycle, where a cycle is a fixed time period, such as 5 minutes. The insulin dose 506 is the insulin delivery for the current cycle.

FIG. 5B depicts an illustrative block diagram 510 for a control system 514 of an exemplary embodiment. In this instance, the control system 514 uses logarithmic glucose level values 512 rather than glucose level values to determine the insulin dose 516. As was mentioned above, the use of the logarithmic glucose level values improves the time in range for the user.

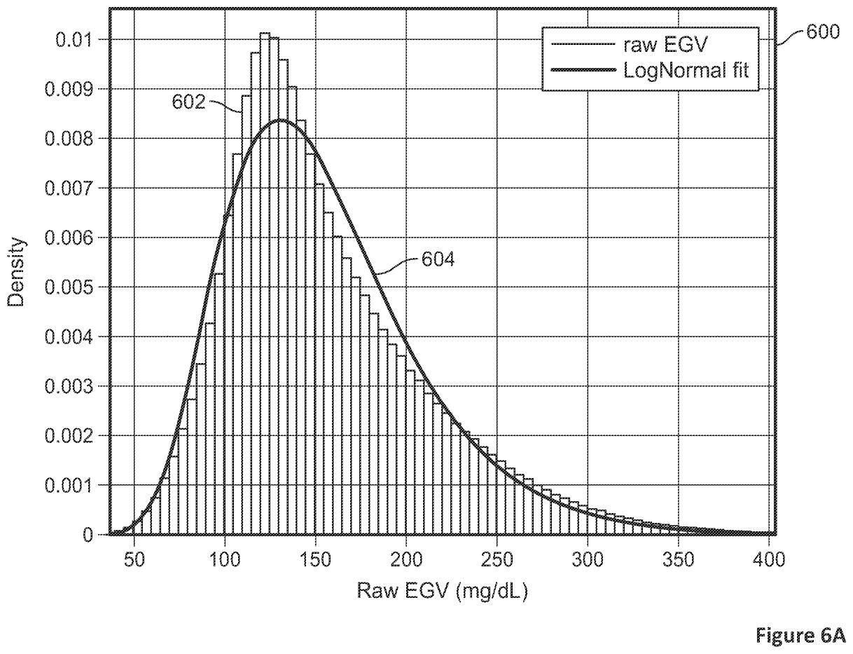

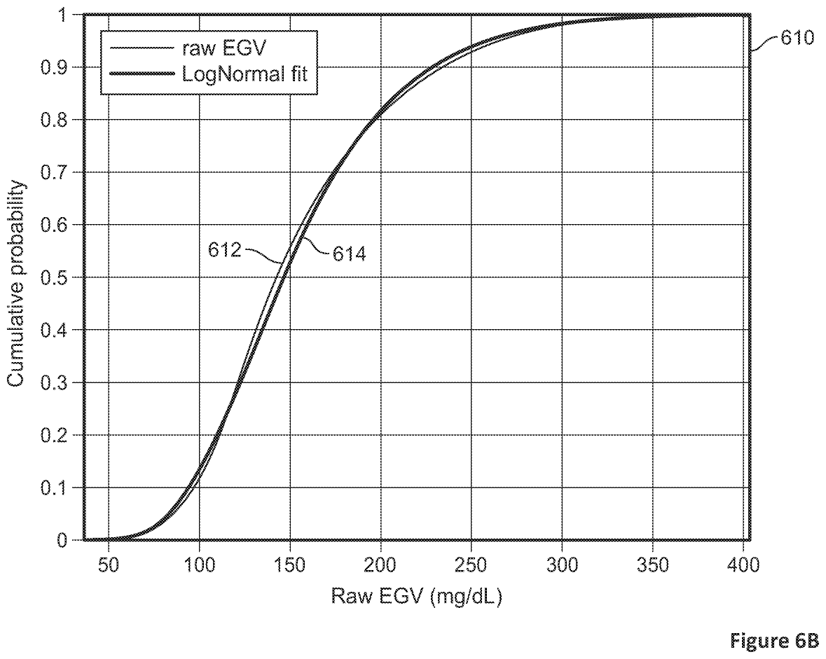

FIG. 6A depicts a plot of density of EGVs. Histogram 602 shows the densities of EGV's for a population. Curve 604 is a curve for a logarithmic normal distribution. As can be seen, the distribution of EGV's appears to conform to a log normal fit (i.e., a logarithmic Gaussian distribution). Hence, the log normal fit appears to be a better match for the EGV's than the normal fit.

The good fit of the log normal fit to the EGV's is also reflected in plot 610 of cumulative probability to EGV's shown in FIG. 6B. Curve 612 is for the cumulative probability of EGV's from the data for the population, and curve 614 is for the cumulative probability for a log normal fit. The two curves 612 and 614 closely correspond.

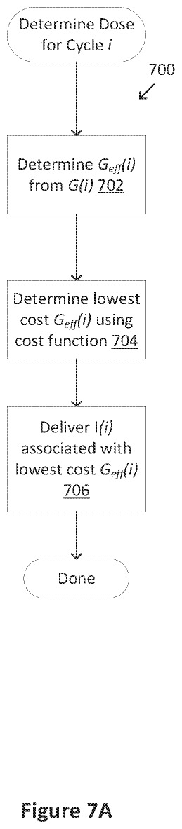

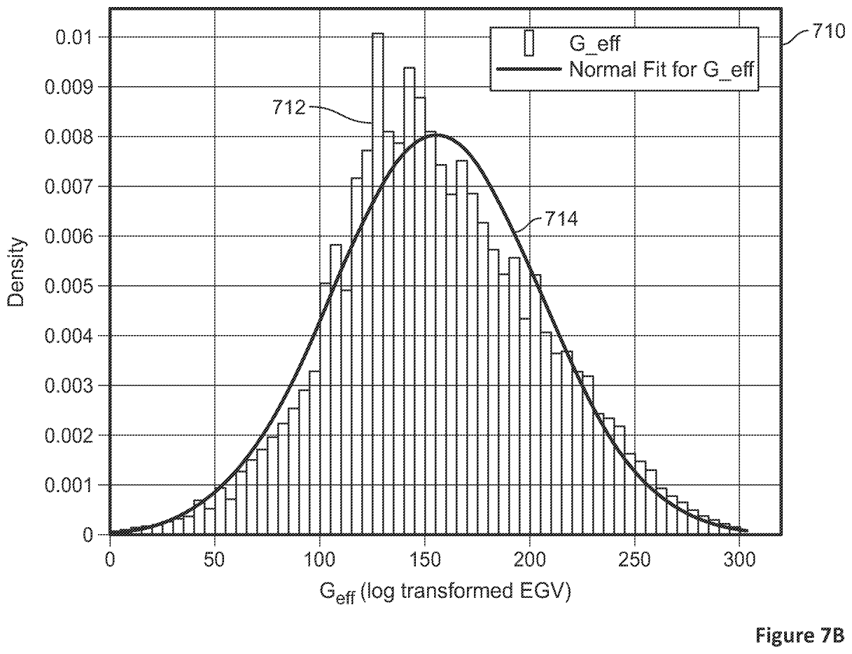

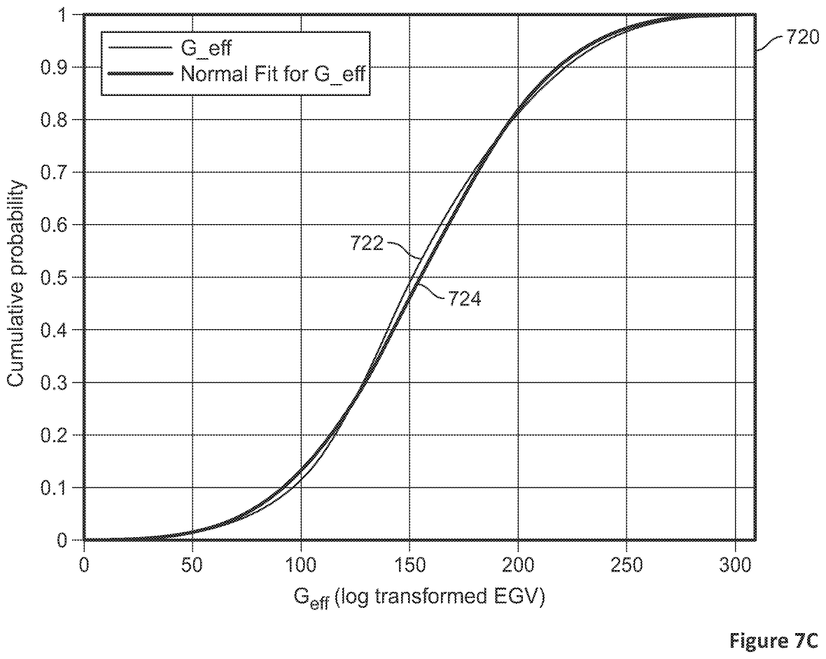

FIG. 7A depicts a flowchart 700 of illustrative steps that may be performed in exemplary embodiments to determine the dose of insulin delivered for a cycle i. At 702, the effective glucose level value, G_{eff}(i), is determined from the glucose level value for cycle i, G(i). The glucose level value G(i) is transformed or filtered to a logarithmic representation that is designated as the effective glucose level value G_{eff}(i), as will be described in more detail below. The transform/filter modifies the glucose level value to a logarithmic value that conforms with a log normal distribution. At 704, a lowest cost G_{eff}(i) is determined using a cost function. Thus, G_{eff}(i) is used in the cost function rather than the conventional approach of using G(i) in the cost function. At 706, the insulin level/(i) that is associated with the lowest cost G_{eff}(i) is delivered to the user by the insulin delivery device.

FIG. 7B depicts a plot 710 of density to G_{eff}(i). Histogram 712 reflects the actual density of various G_{eff}(i) values for the population of users. A normal distribution curve 714 is overlayed over the histogram 712. As can be seen, the histogram 412 largely conforms with a normal distribution of curve 714. FIG. 7C shows a plot 720 of a curve of cumulative probability for G_{eff}(i). The cumulative probability curve 724 for a normal distribution is overlayed. As can be seen, the two curves 722 and 724 are very similar, providing further evidence of G_{eff}(i) conforming to a Gaussian normal distribution.

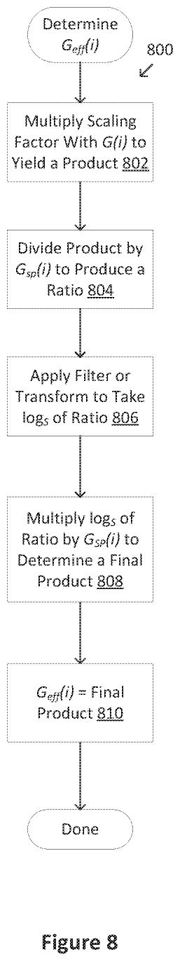

FIG. 8 depicts a flowchart 800 of illustrative steps that may be performed in exemplary embodiments to determine the effective glucose level G_{eff}(i) (see 702). One suitable equation for calculating G_{eff}(i) is:

$${G}_{eff} ( i ) = {G}_{SP} ( i ) \cdot {log}_{2} ( \frac{S \cdot G ( i )}{{G}_{SP} ( i )} ) \qquad ( Eq . 1 )$$

where G_{SP}(i) is the set point glucose level value for control system for cycle i, and S is a scaling factor (that may match the base of the logarithm, e.g., 2). At 802, the glucose level value for cycle i is multiplied by the scaling factor, S. At 804, the resulting product is divided by the set point glucose level Gₛₚ(i) to produce a ratio (S·G(i))/Gₛₚ(i). At 806, a filter or transform is applied to take the log₂ of the ratio. Given equivalencies with logarithms, the log₂ of the ratio equals log₂(S·G(i))-log₂(Gₛₚ(i)). Hence, this is the difference in log₂ of the scaled glucose level value and the target glucose level value. At 808, the log₂ of the ratio is multiplied by the glucose level setpoint Gₛₚ(i) to produce a final product. At 810, G_{eff}(i) is set equal to the final product. Accordingly, in some embodiments, determining the effective glucose level for a respective cycle comprises calculating a glucose level ratio for the respective cycle by multiplying the glucose level value for the respective cycle with a scaling factor and dividing the result by a set point glucose level value for the respective cycle. Subsequently, the effective glucose level for a respective cycle is determined by applying a logarithmic function to the glucose level ratio for the respective cycle and multiplying the result by the set point glucose level for the respective cycle. In some embodiments, the base of the logarithmic function may be 2, Euler's number or 10. In some embodiments, the scaling factor is the same as the base of the logarithmic function.

It should be appreciated that other transforms/filters may be applied to make the glucose level values conform to a normal fit or to another desired type of distribution.

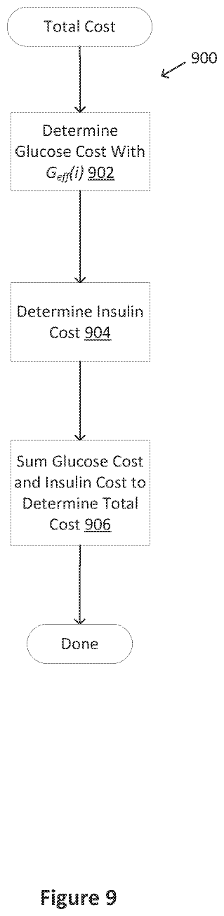

As was mentioned above, a cost function may be used to determine the best insulin dose for a user in a cycle. FIG. 9 depicts a flowchart 900 of illustrative steps that may be performed in exemplary embodiments to determine the total cost of a candidate insulin dose for the cycle. At 902, a glucose cost for the candidate insulin dose using the effective glucose level value G_{eff}(i). An example of how to calculate the glucose cost using G_{eff}(i) is detailed below. The glucose cost is the cost of glucose excursions. At 904, insulin cost for the candidate insulin dose is determined. Insulin cost is the cost of insulin excursions relative a basal insulin standard. At 906, the total cost for the candidate insulin dose is determined by summing the glucose cost and the insulin cost. A suitable cost equation for exemplary embodiments is:

J=Q·Σᵢ₌₁^{M}Gₚ(i)²+R·Σᵢ₌₁ⁿIₚ(i)² (Eq. 2)

where Q and R are weight coefficients as mentioned above, Gₚ(i)² is the square of the deviation between the projected glucose level for an insulin dosage at cycle i and the projected glucose level for the basal insulin dosage, M is the number of cycles in the prediction horizon, Iₚ (i)² is the square of the deviation between the projected insulin delivered at cycle i and the insulin for basal insulin delivery, and n is the control horizon in cycles. Q·Σᵢ₌₁^{M}Gₚ(i)² is the glucose cost and R·Σᵢ₌₁ⁿIₚ (i)² is the insulin cost. Accordingly, in some embodiments determining the total cost for the candidate insulin dose for a cycle comprises summing the glucose cost and the insulin cost. In some embodiments, the glucose cost is determined by summing over a projection horizon the square of the deviation between the projected glucose levels and the projected glucose level for the basal insulin dosage when the candidate insulin is provided to the user at the start of the projection horizon. In some embodiments, the insulin cost is determined by summing over a control horizon the square of the deviation between the projected insulin delivered at the start of the control horizon and the insulin for basal insulin delivery. In some embodiments, the number of cycles in the projection horizon is between about 3 to about 30, more specifically between about 4 to about 20 and in particular between about 5 to about 15. In some embodiments, the number of cycles in the control horizon is between about 3 to about 30, more specifically between about 4 to about 20 and in particular between about 5 to about 15.

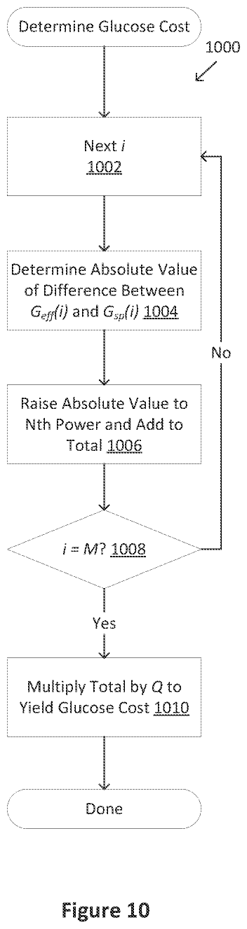

FIG. 10 depicts a flowchart 1000 of illustrative steps that may be performed in exemplary embodiments to determine the glucose cost of a candidate insulin dose for cycle i. A suitable equation is:

Glucose cost=Q·Σᵢ₌₁^{M}|G_{eff}(i)−Gₛₚ(i)|^{N} (Eq. 3)

where N is 1 or 2. At 1002, the cycle index i is incremented. The cycle index may be initialized as 0. At 1004, the absolute value between G_{eff}(i) and Gₛₚ (i) is determined. This calculation captures the magnitude of the difference of the effective glucose level value and the glucose level set point. This difference is raised to the power of N. Where N equals 2, the difference is squared. At 1008, a check is made whether i equals M, which means that all of the cycles in the time horizon have been processed to produce a total. If not, the process repeats beginning at 1002. Otherwise, at 1010 the total is multiplied by a weight Q to produce the glucose cost. Accordingly, in some embodiments, determining the glucose cost comprises determining a deviation factor for a number of projected cycles, wherein determining the deviation factor for each projected cycle comprises the difference between an effective glucose level and a set point glucose level value for a respective cycle and raising the absolute value of the difference by a factor N, wherein N is 1 or 2. Further, determining the glucose cost comprises summing the deviation factors over the number of projected cycles and multiplying the sum by a glucose cost weight coefficient. In some embodiments, the number of projected cycles is between about 3 to about 30, more specifically between about 4 to about 20 and in particular between about 5 to about 15. The number of projected cycles may be the same as the number of cycles in the projection horizon and/or control horizon.

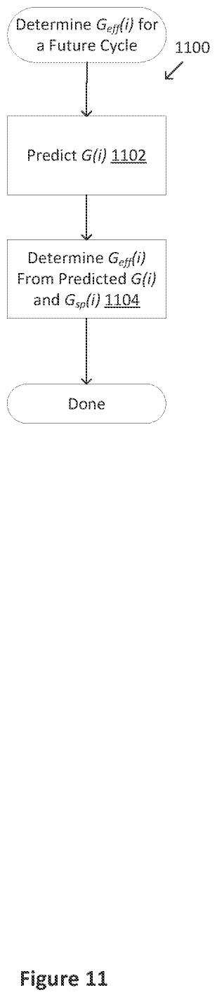

In order to determine G_{eff}(i) for the time horizon values in the future, the exemplary embodiments may perform the steps depicted in the flowchart 1100 of FIG. 11 for each future cycle in the time horizon. Initially, at 1102, the glucose level value for the user for the cycle i is predicted. One suitable approach to predicting glucose value in the current or upcoming cycle, G(i), is to predict based on glucose level values for immediately preceding cycles. The following equation may be used:

G(i)=b₁G(i−1)+b₂G(i−2)+b₃G(i−3) (Eq. 4)

where b₁, b₂, and b₃ are coefficients. Thus, the predicted glucose level values are determined based on the predictions for the immediately preceding cycles for most of the cycles in the time horizon. At 1104, G_{eff}(i) is calculated from predicted G(i) values and the glucose set point values Gₛₚ(i) using, for example, Equation 1. Accordingly, in some embodiments, predicting the glucose value for the current or upcoming cycle comprises multiplying each of a number of glucose values for immediately preceding cycles with a respective coefficient for each glucose value. In some embodiments, the number of glucose values for immediately preceding cycles is between 1 to 5, more specifically between 2 to 4 and in particular 3. In some embodiments, each of the respective coefficient for the glucose values is between 0 to 1.

The present disclosure furthermore relates to computer programs comprising instructions (also referred to as computer programming instructions) to perform the aforementioned functionalities. The instructions may be executed by a processor. The instructions may also be performed by a plurality of processors for example in a distributed computer system. The computer programs of the present disclosure may be for example preinstalled on, or downloaded to the medicament delivery device, management device, fluid delivery device, e.g. their storage.

While exemplary embodiments have been described herein, it should be appreciated that various changes in form and detail may be made without departing from the intended scope of the claims appended hereto.

1. An insulin delivery device for delivering insulin to a user, comprising:
a non-transitory computer-readable storage medium storing computer programming instructions; and
a processor for executing the computer programming instructions to cause the processor to:
determine an insulin dose to be delivered to the user with the insulin delivery device that has a lowest glucose cost, the glucose cost being determined based on effective glucose level values for the user and the effective glucose level values being determined by applying a logarithmic transform to predicted glucose level values for a time horizon, and
cause the insulin dose to be delivered by the insulin delivery device to the user.

2. The insulin delivery device of claim 1, wherein the predicted glucose level values are predicted from previous glucose level values of the user for earlier times and/or from predicted glucose level values for the user for earlier times when previous glucose level values are not yet available.

3. The insulin delivery device of claim 2, wherein the non-transitory computer-readable storage medium stores a glucose level history of the user and wherein the glucose level values of the user for times before an earliest time in the time horizon are part of the glucose level history.

4. The insulin delivery device of claim 1, further comprising an insulin reservoir for holding insulin for delivering the insulin to the user.

5. The insulin delivery device of claim 4, wherein the computer programming instructions, when executed by the processor, cause the processor to initiate delivery of the insulin dose from the reservoir to the user.

6. The insulin delivery device of claim 1, wherein the processor in applying a logarithmic transform to predicted glucose level values for the time horizon applies a logarithmic function to the predicted glucose level values.

7. The insulin delivery device of claim 6, wherein the applying the logarithmic transform comprises, for each of the predicted glucose level values, entails applying a logarithmic function to a product of a scaling factor and a ratio of the predicted glucose level value to the set point glucose level value to yield a logarithmic value.

8. The insulin delivery device of claim 7 wherein the applying the applying the logarithmic transform further comprises multiplying the logarithmic value by the set point glucose level value to produce one of the predicted logarithmic glucose level values.

9. The insulin delivery device of claim 8, wherein the glucose cost is a product of a coefficient and a sum of squared differences between each of the projected glucose level values and the set point glucose level value over the time horizon

10. An insulin delivery system for delivering insulin to a user, comprising:
a non-transitory computer-readable storage medium storing computer programming instructions; and
a processor for executing the computer programming instructions to cause the processor to:
choose a selected one of candidate insulin dosages that has a best cost value when a cost function is applied to the candidate insulin dosages for delivery by the insulin delivery device to the user;
wherein the cost function includes an insulin cost component and a glucose cost component;
wherein the glucose cost component is based on cumulative differences between predicted effective glucose level values and set point glucose level values over a time horizon;
wherein the predicted effective glucose level values are determined by applying a logarithmic function to predicted glucose level values of the user over the time horizon; and
cause delivery of the selected one of the candidate insulin dosages to the user.

11. The insulin delivery system of claim 10, wherein at least one of the predicted glucose level values is determined by the processor from glucose level values of the user for points in time before the time horizon.

12. The insulin delivery system of claim 10, further comprising an insulin reservoir for holding insulin.

13. The insulin delivery system of claim 12, wherein the computer programming instructions, when executed by the processor, cause the processor to initiate delivery of the selected one of the candidate insulin dosages from the reservoir to the user.

14. The insulin delivery system of claim 13, wherein the predicted glucose level values are determined by the processor from glucose level values before the time horizon and/or predicted glucose level values.

15. The insulin delivery system of claim 14, wherein each of the predicted effective glucose level values is determined by applying a logarithmic function to a product of a scaling factor and a ratio of a corresponding predicted glucose level value to one of the set point glucose level values to yield a logarithmic value.

16. The insulin delivery system of claim 15, wherein the computer programming instructions, when executed by the processor, further cause the processor to multiply the logarithmic value by the set point glucose level value to produce one of the predicted logarithmic projected glucose level values.

17. The insulin delivery system of claim 16, wherein the glucose cost value is a product of a coefficient and a sum of squared differences between each of the projected glucose level values and the set point glucose level values over the time horizon.

18. An insulin delivery device, comprising:
a non-transitory computer-readable storage medium storing computer programming instructions; and
a processor for executing the computer programming instructions to cause the processor to:
control insulin delivery to a user by the insulin delivery device, the control including:
transforming a glucose level value of the user into a logarithmic glucose level value;
using the logarithmic glucose level value in determining what insulin dosage is to be delivered to the user by the insulin delivery device; and
causing the determined insulin dosage to be delivered to the user from the insulin delivery device.

19. The insulin delivery device of claim 18, wherein the determined insulin dosage is for basal delivery to the user.

20. The insulin delivery device of claim 18, wherein the transforming comprises applying a logarithmic function to the glucose level value.
